(19) United States
(12) Patent Application Publication
(10) Pub. No.: US 2013/0331836 A1
(43) Pub. Date: Dec. 12, 2013

(54) ELECTROSURGICAL INSTRUMENT

(71) Applicant: GYRUS MEDICAL LIMITED, Cardiff (GB)

(72) Inventor: Richard John KEOGH, Caerphilly (GB)

(73) Assignee: CYRUS MEDICAL LIMITED, Cardiff (GB)

(21) Appl. No.: 13/915,078

(22) Filed: Jun. 11, 2013

(30) Foreign Application Priority Data
Jun. 12, 2012 (GB) 1210298.4

(51) Int. Cl. A61B 18/14

(52) U.S. Cl. CPC A61B 18/1482
USPC 606/41

(57) ABSTRACT

An electrosurgical instrument is provided for the treatment of tissue, the instrument (3) comprising an instrument shaft (14) having a longitudinal axis, a suction lumen (24) extending at least partially along the instrument shaft, and an electrode assembly at one end of the shaft. The electrode assembly comprises at least one tissue treatment electrode (16), the tissue treatment electrode having an end face (17) and at least one side face (18). The tissue treatment electrode includes a plurality of apertures in communication with the suction lumen, at least one aperture (19) being present in the end face and at least one aperture (20) being present in the side face.

### CROSS REFERENCE TO RELATED APPLICATIONS

This application claims priority from U.K. Patent Application No. 1210298.4, filed on Jun. 12, 2012, the entire contents of which are incorporated herein by reference.

### TECHNICAL FIELD

This invention relates to an electrosurgical instrument for the treatment of tissue. Such instruments are commonly used for the vaporisation and/or coagulation of tissue in surgical intervention, most commonly in “keyhole” or minimally invasive surgery, but also in “open” surgery.

### BACKGROUND TO THE INVENTION

There is a frequent requirement during a surgical procedure for suction in order to remove matter from the surgical site, whether it is tissue debris, smoke, fluid, gas bubbles or other unwanted matter that interfere with the procedure or obscure the surgeon's view of the surgical site. U.S. Pat. Nos. 6,210,405 & 6,482,202 describe examples of this type of surgical instrument, and it is the object of the present invention to provide an improvement to such suction instruments.

### SUMMARY OF INVENTION

Accordingly, from one aspect an electrosurgical instrument is provided for the treatment of tissue, the instrument comprising an instrument shaft having a longitudinal axis, a suction lumen extending at least partially along the instrument shaft, and an electrode assembly at one end of the shaft, the electrode assembly comprising at least one tissue treatment electrode, the tissue treatment electrode having a distal end face with at least a transverse portion oriented at a 90 degree angle to the longitudinal axis, and at least one side face with at least a radial portion offset but parallel to the longitudinal axis, the tissue treatment electrode including a plurality of apertures in communication with the suction lumen, at least one aperture being present in the transverse portion of the distal end face so as to be capable of aspirating material in the region of the distal end face of the electrode, and at least one aperture being present in the radial portion of the side face so as to be capable of aspirating material in the region of the side face of the electrode.

The instrument of the present invention can provide suction both through the end face of the tissue treatment electrode and through the side face thereof. This means that whatever the orientation of the instrument tip, the surgeon can achieve the effective evacuation of debris from the surgical site. Furthermore, the provision of different suction apertures means that should one aperture become obscured, either by tissue or by debris too large to pass through the aperture, other apertures are still available to to provide effective suction.

According to one convenient arrangement, the tissue treatment electrode is substantially cylindrical with a side face that is circular in cross-section. The tissue treatment electrode is preferably provided with a plurality of apertures disposed around the side face of the tissue treatment electrode. This means that there is suction available around a large area of the instrument tip, preferably throughout 360 degrees. Conveniently, the apertures are equally spaced around the side face of the tissue treatment electrode.

Conveniently, the end face of the tissue treatment electrode is substantially curved, typically in the form of a hemisphere. The end face of the tissue treatment electrode is conveniently provided with a single aperture located at the centre of the end face. In this way, the end face of the tissue treatment electrode has an effective surface are for the treatment of tissue, but still provides suction to evacuate tissue or other debris from the surgical site.

Preferably, there is a discrete transition between the end face and the side face of the electrode. In this way, the user of the surgical instrument is able to select either the end face or the side face as the tissue treatment area of the electrode, as required. Thus, the user may orient the electrode relative to the tissue to be treated such that only the end face is in contact with the tissue. Conversely, the user may orient the electrode relative to the tissue to be treated such that only the side face is in contact with the tissue. Either way, the user has more control over the tissue treatment effect of the electrode, as compared with prior art designs where the side and end faces merge one into the other. Conveniently, the transition is an annular elbow having an angle between the side and end faces of at least 30 degrees.

According to a preferred arrangement, the electrode assembly includes a return electrode separated from the tissue treatment electrode by an insulator therebetween. This allows the use of the electrode assembly as a bipolar system, with the advantages known to be associated with bipolar systems. The return electrode is preferably axially set back with respect to the tissue treatment electrode.

In addition, an embodiment of the invention also provides an electrosurgical instrument, comprising an instrument shaft, a suction lumen extending at least partially along the length of the instrument shaft, and an electrode assembly located at one end of the instrument shaft. The electrode assembly comprises a cupola-shaped tissue treatment electrode having at least one side wall and an end face, the at least one side wall having one or more suction apertures located therein, and the end face having at least one suction aperture located therein, the suction apertures being in fluid communication with the suction lumen for the aspiration of material in the region of said suction apertures.

The cupola-shaped tissue treatment electrode provided with suction apertures on the end face and the side wall provides for convenient and effective evacuation of debris from the surgical site. In particular, the provision of suction apertures on different faces of the cupola-shaped tissue treatment electrode means that blockage of all of the apertures is unlikely, and aspiration should therefore be maintained even if the suction apertures are blocked on at least one face of the cupola.

In one embodiment a plurality of suction apertures are provided in the at least one side wall of the cupola-shaped tissue treatment electrode. In particular, preferably the plurality of suction apertures are substantially equiangularly arranged around the at least one side wall of the cupola-shaped tissue treatment electrode. Providing multiple suction apertures increases aspiration and prevents the chance of all apertures being blocked, if only temporarily.

In an embodiment a single suction aperture is provided in the end face of the cupola-shaped tissue treatment electrode, and preferably the single suction aperture is substantially centrally located in the end face of the cupola-shaped tissue treatment electrode.

In addition, in an embodiment the end face of the cupola-shaped tissue treatment electrode may be angled with respect to the side wall. More preferably the end face of the tissue treatment electrode may also be curved in shape, and even more preferably dome-shaped. Where the electrode is dome shaped and a single suction aperture is provided then the aperture is at the apex of the dome.

Conveniently the cupola-shaped tissue treatment electrode is substantially circular in cross-section, the cross-section being transverse to the instrument shaft. This provides for a cylindrical cupola, preferably with a dome shaped end face.

The electrosurgical instrument may further comprise a return electrode axially separated from the tissue treatment electrode along the instrument shaft. The return electrode provides a return current path for electrical current. The return electrode is conveniently substantially cylindrical in shape.

The electrode can be part of an electrosurgical system including an electrosurgical instrument and an electrosurgical generator, the electrosurgical instrument including an instrument shaft having a longitudinal axis, a suction lumen extending at least partially along the instrument shaft, and an electrode assembly at one end of the shaft, the electrode assembly comprising at least one tissue treatment electrode and at least one return electrode, each of the electrodes being electrically insulated one from another by means of one or more insulation members, the tissue treatment electrode having an end face and at least one side face, the tissue treatment electrode including a plurality of apertures in communication with the suction lumen, at least one aperture being present in the end face and at least one aperture being present in the side face.

In a first arrangement, the generator and electrosurgical instrument are such that the instrument is designed to be operated in a conductive fluid, with the conductive fluid completing the current path between the electrodes. This means that the system operates to perform what is known as “underwater” electrosurgery, in which the conductive site is immersed in a conductive fluid such as saline, and the electrodes operate immersed in said conductive fluid. An example of this type of electrosurgical system is given in our earlier U.S. Pat. No. 6,004,319. The power and voltage setting used by the generator are such that the conductive fluid surrounding the electrodes is vaporised when the electrosurgical instrument is operated in its cutting mode. The provision of suction apertures, not only in the end face of the tissue treatment electrode but also in the side face thereof, allows for fluid, to be evacuated from the surgical site, helping to ensure that the fluid is replenished with a fresh supply of conductive fluid. Other debris, such as vaporised tissue particles, gas bubbles, and other matter can be evacuated from the surgical site through the apertures, to help maintain the visibility of the surgical site.

Alternatively, the generator and electrosurgical instrument are such that the instrument is designed to be operated in a dry-field environment, with the electrodes being in direct contact with the tissue to be treated, and with the tissue completing the current path therebetween. An example of this type of electrosurgical system is given in our earlier U.S. Pat. No. 6,832,998. The power and voltage settings used by the generator are generally lower than in underwater electrosurgical systems, as the electrodes contact the tissue directly and there is no need to form a pocket of vaporised saline surrounding the electrode. In this instance, the suction apertures are used for the evacuation of tissue particles or smoke particles, both of which can obscure the field of view if not removed from the surgical site.

### DESCRIPTION OF THE DRAWINGS

The invention will now be further described, by way of example only, with reference to the accompanying drawings, in which:

FIG. 1 is a schematic diagram of an electrosurgical system using an electrosurgical instrument in accordance with the present invention,

FIG. 2 is a perspective view of the tip of an electrosurgical instrument in accordance with the present invention and capable of being used in the system of FIG. 1, and

FIG. 3 is a cut-away perspective view of the tip of FIG. 2.

### DESCRIPTION OF EMBODIMENTS

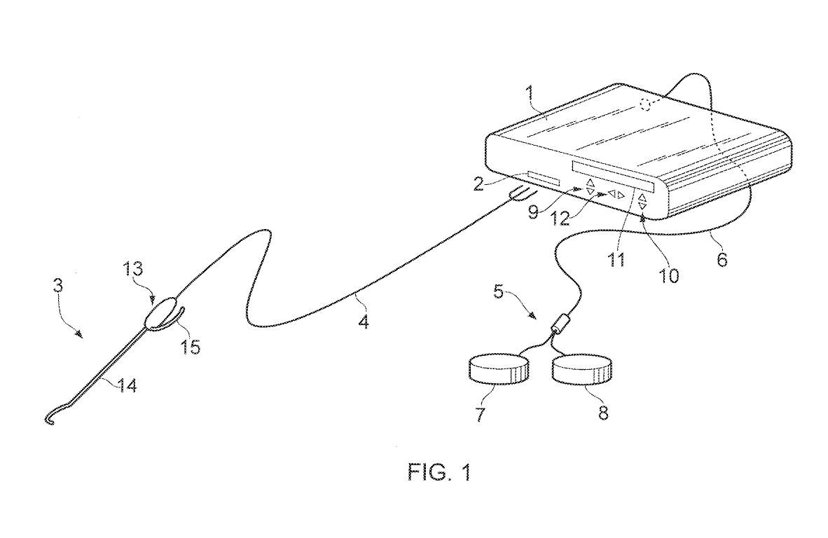

Referring to the drawings, FIG. 1 shows electrosurgical apparatus including a generator 1 having an output socket 2 providing a radio frequency (RF) output, via a connection cord 4, for an electrosurgical instrument 3. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 7 and 8 for selecting a desiccation mode and a vaporisation mode of the generator 1 respectively. The generator front panel has push buttons 9 and 10 for respectively setting desiccation and vaporisation power levels, which are indicated in a display 11. Push buttons 12 are provided as an alternative means for selection between the desiccation and vaporisation modes.

The electrosurgical instrument 3 comprises a housing 13 with an elongate shaft 14, and tissue treatment electrodes at the distal end of the shaft, as will be described below. A movable handle 15 associated with the housing can be actuated to cause the shaft to bend. This instrument is particularly suited to the treatment of the hip joint, where a relatively long shaft with articulation capability is needed to access the area to the treated.

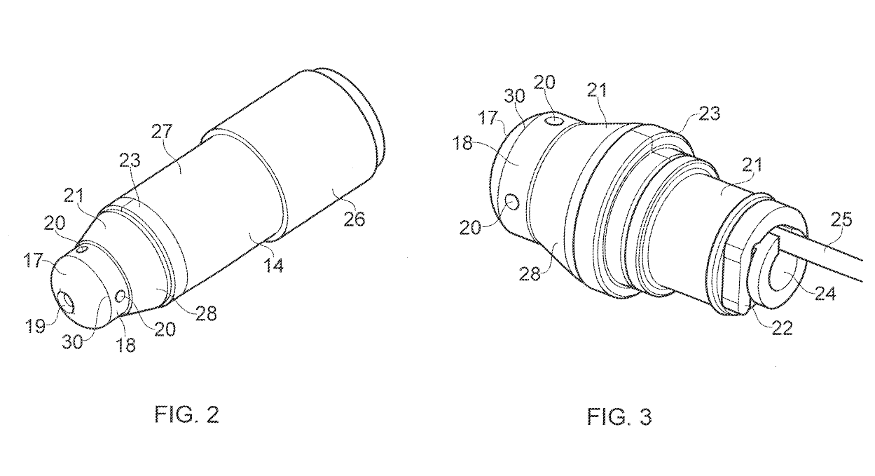

FIGS. 2 & 3 show a tissue treatment electrode 16 comprising a hemispherical end face 17 and a cylindrical side face 18. The electrode is typically formed from tungsten (or an alloy of tungsten and platinum), and can be formed from a single integral component or from two components welded one to the other. Whether formed from one or two components, the end face 17 meets the side face 18 at a discrete transition 30, in the form of an elbow portion at which the end face 17 and side face 18 meet at an angle of at least 30 degrees. That is, the end face 17 and the side face 18 with the discrete transition 30 therebetween together form a cupola-shaped electrode, with the side face 18 forming the side walls of the cupola, and the end face 17 forming the cap or “roof” of the cupola. The end face 17 is provided with a single aperture 19 located at the centre thereof, while the side face 18 is provided with a plurality of apertures 20 spaced at equal distances around its circumference. The tissue treatment electrode is located on a ceramic component 21, and held in place by a c-clip 22. A split ring retainer 23 is then placed over the ceramic component 21, and the end of the shaft 14 is laser-welded to the split ring 23. The ceramic component 21 is hollow so as to form a suction lumen 24, and also to accommodate a lead 25 to supply RF energy to the tissue treatment electrode 16.

The majority of the shaft 14 is covered by an insulating sheath 26, but a portion is left uncovered in order to form a return electrode 27. The return electrode 27 is separated from the tissue treatment electrode 16 by a portion of the ceramic component, forming an insulator 28. In use the instrument 3 is introduced into the body of a patient and manoeuvred into position, typically adjacent a hip joint. RF energy is supplied to the tissue treatment electrode 16, which is used to vaporise or coagulate tissue depending on the type of RF energy supplied from the generator 1. The instrument is typically used submerged in an electrically conductive fluid, such as normal saline, with the RF energy flowing from the tissue treatment electrode 16, through the conductive liquid to the return electrode 27, with any tissue entering the region of the tissue treatment electrode 16 being vaporised or coagulated depending on the circumstances.

The suction lumen 24 is connected to a source of suction, and the apertures 19 and 20 are in communication with the suction lumen 24. In this way, any tissue fragments, bubbles or other debris in the vicinity of the tissue treatment electrode 16 will be drawn into the suction lumen via either the aperture 19 in the end face 17 or the multiple apertures 20 in the side face 18. The provision of apertures in both the end face 17 and the side face 18 allows for debris to be removed from the vicinity of the tissue treatment electrode regardless of its orientation with respect thereto. The multiple suction apertures also allow for the suction effect to be maintained even if one aperture is temporarily blocked by a large tissue fragment or because that portion of the tissue treatment electrode is embedded in the tissue to be treated. The fact that the apertures 19 & 20 are located directly in the tissue treatment electrode 16, rather than in the insulator 28 or any other component, means that the electrode 16 can be used to vaporise any tissue blocking the apertures 19 & 20, and hence render them effective once again. In this way, the instrument is designed to ensure that the evacuation of tissue fragments and other debris remains effective, whatever the size and whatever the position of the debris with respect to the electrode 16.

As previously mentioned, the instrument 3 is primarily designed to be operated in a conductive fluid such as saline, with the fluid completing the circuit between the electrodes. However, the instrument 3 can also be used as a dry-field instrument, in which case the user must ensure that the electrodes are placed in contact with the tissue to be treated. In this way, the current flows from the tissue treatment electrode, through the tissue, to the return electrode 27.
Alternative embodiments will be envisaged by those skilled in the art without departing from the scope of the present invention. For example, the electrosurgical instrument can also be used for delivering a blended power output. This is achieved by automatically alternating the output of the RF generator 1 between the coagulation and vaporisation power levels, so that more haemostasis is produced then is possible in the vaporisation mode. As a consequence, the speed of tissue debulking is reduced, but the increased haemostasis is useful when cutting or debulking vascular tissue structures. Alternatively, the output of the RF generator 1 can be pulsed at the vaporisation power level, without cycled activation of the coagulation mode. This produces a less aggressive tissue vaporisation than occurs in the vaporisation mode, with a consequent reduction in both bubble formation and the risk of tissue charring.

1. An electrosurgical instrument for the treatment of tissue, the instrument comprising an instrument shaft having a longitudinal axis, a suction lumen extending at least partially along the instrument shaft, and an electrode assembly at one end of the shaft, the electrode assembly comprising at least one tissue treatment electrode, the tissue treatment electrode having a distal end face with at least a transverse portion oriented at a 90 degree angle to the longitudinal axis, and at least one side face with at least a radial portion offset but parallel to the longitudinal axis, the tissue treatment electrode including a plurality of apertures in communication with the suction lumen, at least one aperture being present in the transverse portion of the distal end face so as to be capable of aspirating material in the region of the distal end face of the electrode, and at least one aperture being present in the radial portion of the side face so as to be capable of aspirating material in the region of the side face of the electrode.

2. An electrosurgical instrument according to claim 1, wherein the tissue treatment electrode is substantially cylindrical with a side face that is circular in cross-section.

3. An electrosurgical instrument according to claim 2, wherein the tissue treatment electrode is provided with a plurality of apertures disposed around the side face of the tissue treatment electrode.

4. An electrosurgical instrument according to claim 3, wherein the apertures are equally spaced around the side face of the tissue treatment electrode.

5. An electrosurgical instrument according to claim 1, wherein the end face of the tissue treatment electrode is substantially curved.

6. An electrosurgical instrument according to claim 5, wherein the end face of the tissue treatment electrode is substantially in the form of a hemisphere.

7. An electrosurgical instrument according to claim 1, wherein the end face of the tissue treatment electrode is provided with a single aperture located at the centre of the end face.

8. An electrosurgical instrument according to claim 1, wherein there is a discrete transition between the end face and the side face of the electrode.

9. An electrosurgical instrument according to claim 8, wherein the transition is an annular elbow portion having an angle between the side and end faces of at least 30 degrees.

10. An electrosurgical instrument according to claim 1, wherein the electrode assembly includes a return electrode separated from the tissue treatment electrode by an insulator therebetween.

11. An electrosurgical instrument according to claim 10, wherein the return electrode is axially set back with respect to the tissue treatment electrode.

12. An electrosurgical instrument, comprising:
an instrument shaft;
a suction lumen extending at least partially along the length of the instrument shaft; and
an electrode assembly located at one end of the instrument shaft;
the electrode assembly comprising a cupola-shaped tissue treatment electrode having at least one side wall and an end face, the at least one side wall having one or more suction apertures located therein, and the end face having at least one suction aperture located therein, the suction apertures being in fluid communication with the suction lumen for the aspiration of material in the region of said suction apertures.

13. An electrosurgical instrument according to claim 12, wherein a plurality of suction apertures are provided in the at least one side wall of the cupola-shaped tissue treatment electrode.

14. An electrosurgical instrument according to claim 13, wherein the plurality of suction apertures are substantially equiangularly arranged around the at least one side wall of the cupola-shaped tissue treatment electrode.

15. An electrosurgical instrument according to claim 12, wherein a single suction aperture is provided in the end face of the cupola-shaped tissue treatment electrode.

16. An electrosurgical instrument according to claim 15, wherein the single suction aperture is substantially centrally located in the end face of the cupola-shaped tissue treatment electrode.

17. An electrosurgical instrument according to claim 12, wherein the end face of the cupola-shaped tissue treatment electrode is angled with respect to the side wall, and more preferably curved in shape, and even more preferably dome-shaped.

18. An electrosurgical instrument according to claim 12, wherein the cupola-shaped tissue treatment electrode is substantially circular in cross-section, the cross-section being transverse to the instrument shaft.

19. An electrosurgical instrument according to claim 12, and further comprising a return electrode axially separated from the tissue treatment electrode along the instrument shaft.

20. An electrosurgical instrument according to claim 19, wherein the return electrode is substantially cylindrical in shape.
